(19) United States
(12) Patent Application Publication
(10) Pub. No.: US 2026/0027374 A1
(43) Pub. Date: Jan. 29, 2026

(54) Electrode Implementation for Enhanced Tissue Contact on IMD

(71) Applicant: Cardiac Pacemakers, Inc., St Paul, MN (US)

(72) Inventors: Jean M. Bobgan, Maple Grove, MN (US); Peter Joyce, Nenagh (IE); James M. English, Cahir (IE)

(21) Appl. No.: 19/277,990

(22) Filed: Jul. 23, 2025

Related U.S. Application Data
(60) Provisional application No. 63/676,037, filed on Jul. 26, 2024.

(51) Int. Cl. A61N 1/375

(52) U.S. Cl. CPC A61N 1/3756

(57) ABSTRACT

An implantable medical device includes multiple end electrodes. The implantable medical device includes a first end surface formed by a first end electrode and a second end surface formed by a second end electrode. The first end electrode and the second end electrode are positioned at opposite ends of the implantable medical device.

### CROSS REFERENCE TO RELATED APPLICATION

This application claims priority to Provisional Application No. 63/676,037, filed Jul. 26, 2025, which is herein incorporated by reference in its entirety.

### TECHNICAL FIELD

Instances of the present disclosure relate to medical devices and systems for sensing physiological parameters. More specifically, instances of the present disclosure relate to implantable medical devices with one or more electrodes that promote tissue contact.

### BACKGROUND

Implantable medical devices may include one or more electrodes to assist with sensing physiological parameters such as cardiac activation signals.

### SUMMARY

In Example 1, an implantable medical device including: a first housing section having a first major surface and a first end surface; a first end electrode that forms the first end surface; a second housing section opposite the first housing section in a longitudinal direction of the implantable medical device, the second housing section having a second major surface and a second end surface; and a second end electrode that forms the second end surface.

In Example 2, the implantable medical device of Example 1, further including: a first leading edge and a second leading edge. The implantable medical device extends along the longitudinal direction between the first leading edge and the second leading edge. The first end electrode is wrapped around the first leading edge. The second end electrode is wrapped around the second leading edge.

In Example 3, the implantable medical device of Example 1, wherein the first end electrode and the second end electrode each comprise: a curved edge portion; a face electrode portion to cover at least a portion of the first or second major surface; and a transition portion to connect the curved edge portion to the face electrode portion.

In Example 4, the implantable medical device of Example 3, wherein the transition portion is curved.

In Example 5, the implantable medical device of any of Examples 1-4, wherein the first housing section comprises a ceramic material.

In Example 6, the implantable medical device of any of Examples 1-5, further including a plurality of first porous features formed on the first end electrode.

In Example 7, the implantable medical device of Example 6, further including a plurality of second porous features formed on the second end electrode.

In Example 8, the implantable medical device of Example 6, further including a plurality of second porous features formed on an area at least partially surrounding at least one of the first end electrode and the second end electrode.

In Example 9, a method of making an implantable medical device includes: forming a first housing section having a first major surface and a first end surface; forming a first end electrode at least on the first end surface; forming a second housing section opposite the first housing section in a longitudinal direction of the implantable medical device, the second housing section having a second major surface and a second end surface; and forming a second end electrode at least on the second end surface.

In Example 10, the method of Example 9, wherein the implantable medical device extends along the longitudinal direction between a first leading edge and a second leading edge, the first end electrode is formed to wrap around the first leading edge, and the second end electrode is formed to wrap around the second leading edge.

In Example 11, the method of Example 10, further including forming respective porous surface areas on the first end electrode and the second end electrode.

In Example 12, an implantable medical device includes: a first housing section having a first major surface and a first end surface; an end electrode that forms the first end surface; a second housing section opposite the first housing section in a longitudinal direction of the implantable medical device, the second housing section having a second major surface and a second end surface; and a face electrode that covers a portion of the second major surface but not the second end surface. The face electrode forms a shape that protrudes away from the second major surface to increase surface area of the face electrode exposed to tissue.

In Example 13, the implantable medical device of Example 12, wherein the shape is a dome that forms a side profile with a linear slope portion as the face electrode extends from the first major surface towards a peak of the face electrode.

In Example 14, the implantable medical device of Example 13, wherein the side profile includes a curved transition between the linear slope portion and the peak.

In Example 15, the implantable medical device of Example 12, wherein the shape is a dome that forms a side profile that continuously curves between the first major surface and a peak of the face electrode.

In Example 16, an implantable medical device includes: a first housing section having a first major surface and a first end surface; a first end electrode that forms the first end surface; a second housing section opposite the first housing section in a longitudinal direction of the implantable medical device, the second housing section having a second major surface and a second end surface; and a second end electrode that forms the second end surface.

In Example 17, the implantable medical device of Example 16, further including: a first leading edge and a second leading edge. The implantable medical device extends along the longitudinal direction between the first leading edge and the second leading edge. The first end electrode is wrapped around the first leading edge. The second end electrode is wrapped around the second leading edge.

In Example 18, the implantable medical device of Example 16, wherein the first end electrode and the second end electrode each comprise: a curved edge portion; a face electrode portion to cover at least a portion of the first or second major surface; and a transition portion to connect the curved edge portion to the face electrode portion.

In Example 19, the implantable medical device of Example 18, wherein the transition portion is curved.

In Example 20, the implantable medical device of Example 16, wherein the first housing section comprises a ceramic material.

In Example 21, the implantable medical device of Example 16, further including a plurality of first porous features formed on the first end electrode.

In Example 22, the implantable medical device of Example 21, further including a plurality of second porous features formed on the second end electrode.

In Example 23, the implantable medical device of Example 21, further including a plurality of second porous features formed on an area at least partially surrounding at least one of the first end electrode and the second end electrode.

In Example 24, the implantable medical device of Example 1, wherein first end electrode and the second electrode comprise titanium.

In Example 25, an implantable medical device includes: a first housing section having a first major surface and a first end surface; an end electrode that forms the first end surface; a second housing section opposite the first housing section in a longitudinal direction of the implantable medical device, the second housing section having a second major surface and a second end surface; and a face electrode that covers a portion of the second major surface but not the second end surface. The face electrode forms a shape that protrudes away from the second major surface to increase surface area of the face electrode exposed to tissue.

In Example 26, the implantable medical device of Example 25, wherein the dome forms a side profile with a linear slope portion as the face electrode extends from the first major surface towards a peak of the face electrode.

In Example 27, the implantable medical device of Example 26, wherein the side profile includes a curved transition between the linear slope portion and the peak.

In Example 28, the implantable medical device of Example 25, wherein the dome forms a side profile that continuously curves between the first major surface and a peak of the face electrode.

In Example 29, the implantable medical device of Example 28, wherein the side profile has a first slope and a second slope, wherein the second scope decreases as the face electrode extends from the first major surface towards the peak.

In Example 30, the implantable medical device of Example 25, wherein the shape comprises a truncated cone shape extending between the first major surface and an outermost surface for direct tissue contact.

In Example 31, the implantable medical device of Example 25, further including protrusions extending from the second major surface.

In Example 32, the implantable medical device of Example 31, wherein the face electrode has a peak at or higher than a distal tip of the protrusions.

In Example 33, a method of making an implantable medical device includes: forming a first housing section having a first major surface and a first end surface; forming a first end electrode at least on the first end surface; forming a second housing section opposite the first housing section in a longitudinal direction of the implantable medical device, the second housing section having a second major surface and a second end surface; and forming a second end electrode at least on the second end surface.

In Example 34, the method of Example 33, wherein: the implantable medical device extends along the longitudinal direction between a first leading edge and a second leading edge, the first end electrode is formed to wrap around the first leading edge, and the second end electrode is formed to wrap around the second leading edge.

In Example 35, the method of Example 33, further including: forming respective porous surface areas on the first end electrode and the second end electrode.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting a patient monitoring system, in accordance with certain instances of the present disclosure.

FIG. 2A is a side view of an implantable medical device, in accordance with certain instances of the present disclosure.

FIG. 2B is a simplified schematic diagram of an implantable medical device, in accordance with certain instances of the present disclosure.

FIGS. 3A-3B are side views of an end electrode of an implantable medical device, in accordance with certain instances of the present disclosure.

FIG. 4 is a side view of an implantable medical device, in accordance with certain instances of the present disclosure.

FIGS. 5A-5C are different views of a portion of the implantable medical device of FIG. 4, in accordance with certain instances of the present disclosure.

FIG. 5D is a side view of a porous electrode surface of the electrode of FIG. 5C, in accordance with certain instances of the present disclosure.

FIG. 6 shows example electrical connections of an electrode of an outer housing section for use with medical devices, in accordance with certain instances of the present disclosure.

FIG. 7 depicts a block diagram of a method of manufacturing or making an implantable medical device described herein, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

When a medical device with electrodes is implanted subcutaneously within a patient's body (e.g., within a pocket in the patient's chest or abdomen), surrounding tissue will begin to contact the electrodes. Increasing the quality of tissue-to-electrode contact can reduce relative movement of the implanted medical device with respect to the tissue after implantation-which can increase the quality of measurements taken using the electrodes. Poor tissue-to-electrode contact may cause fluctuating measurements associated with the implanted medical device (e.g., abnormally high and fluctuating impedance measurements).

Certain instances of the present disclosure utilize a configuration of one or more electrodes on an outer surface of an implantable medical device that promotes tissue-electrode contact after implantation. This can assist with reducing fluctuations of measurements associated with the implantable medical device (e.g., impedance measurements) and reducing the relative movement of the implantable medical device with respect to surrounding tissue. As one example, forming electrodes on opposite end of an implanted medical device, the distance between the electrodes is greater than if one of the electrodes was a face electrode (e.g., not positioned at one of the distal ends of the IMD 200), and increasing the distance between the electrodes can improve the quality of the measurements sensed using the electrodes. As another example, using two end electrodes increases the overall surface area of the electrodes as compared to if one of the electrodes was a face electrode, and increasing the surface area can further improve the quality of the measurements sensed using the electrodes. As another example, because ends of an implanted medical device are typically under a more constant pressure load (as compared to a device with only one end electrode), an implanted medical device is more likely to be in consistent contact with tissue. Similar benefits can be realized with an implanted medical device with an end electrode and a face electrode that protrudes from an implanted medical device.

### System

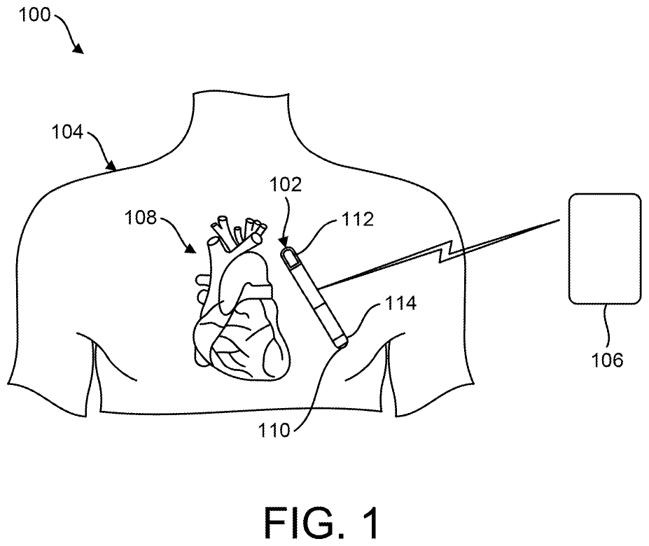

FIG. 1 is a schematic illustration of a system 100 including an implantable medical device (IMD) 102 implanted within a patient's body 104 and configured to communicate with a receiving device 106. The IMD 102 can be implanted subcutaneously within an implantation location or pocket in the patient's chest or abdomen and can be configured to monitor (e.g., sense and/or record) physiological parameters associated with the patient's heart 108. The IMD 102 can be an implantable cardiac monitor (e.g., an implantable diagnostic monitor, an implantable loop recorder) configured to record physiological parameters such as, for example, one or more cardiac activation signals, heart sounds, blood pressure measurements, oxygen saturations. Further, the IMD 102 can be configured to monitor physiological parameters that may include one or more signals indicative of a patient's physical activity level and/or metabolic level, such as an acceleration signal.

For purposes of illustration, and not of limitation, various instances of devices that may be used to record physiological parameters in accordance with the present disclosure are described herein in the context of IMDs that may be implanted under the skin in the chest region of a patient. However, the IMD 102 may include any type of IMD, any number of different components of an implantable system, and/or the like having a housing and being configured to be implanted in a patient's body 104. For example, the IMD 102 can include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the patient's body. In various instances, the IMD 102 can include both defibrillation and pacing/CRT capabilities (e.g., a CRT-D device).

As shown, the IMD 102 includes a housing 110 having two electrodes 112 and 114 coupled thereto—although a different number of electrodes can be used with the IMD 102. As will be described in greater detail, the electrodes 112 and 114 are positioned at opposite leading edges of the housing 110 along a longitudinal axis of the IMD 102. The first electrode 112 is wrapped around a first leading edge of the housing 110, and the second electrode 114 is wrapped around a second leading edge of the housing 110 which is opposite the first leading edge. As such, the IMD 102 includes two end electrodes (e.g., electrodes that form respective end surfaces of the housing). The electrodes 112 and 114 can each have a surface configuration to provide a tissue pressure profile thereon to promote tissue contact.

The IMD 102 can be configured to sense and record physiological parameters. For example, the IMD 102 may be configured to activate (e.g., periodically, continuously, upon detection of an event, and/or the like), record a specified amount of data (e.g., physiological parameters) in a memory, and communicate that recorded data to the receiving device 106. The receiving device 106 may be, for example, a programmer, controller, patient monitoring system, and/or the like. Although illustrated in FIG. 1 as an external device, the receiving device 106 can include an implantable device configured to communicate with the IMD 102 that can, for example, be a control device, another monitoring device, a pacemaker, an ICD, a CRT device, and/or the like.

The IMD 102 and the receiving device 106 can communicate through a wireless link. For example, as will be described in more detail below, the IMD 102 can include an antenna, which transmits and/or receives signals from the receiving device 106. The IMD 102 and the receiving device 106 can be communicatively coupled through a short-range communications link, such as Bluetooth, IEEE 802.11, and/or a proprietary wireless protocol. The communications link can facilitate uni-directional and/or bi-directional communication between the IMD 102 and the receiving device 106. Data and/or control signals can be transmitted between the IMD 102 and the receiving device 106 to coordinate the functions of the IMD 102 and/or the receiving device 106. The patient data can be downloaded from one or more of the IMD 102 and the receiving device 106 periodically or on command. The physician and/or the patient may communicate with the IMD 102 and the receiving device 106, for example, to acquire patient data or to initiate, terminate, or modify recording and/or therapy.

### Medical Device

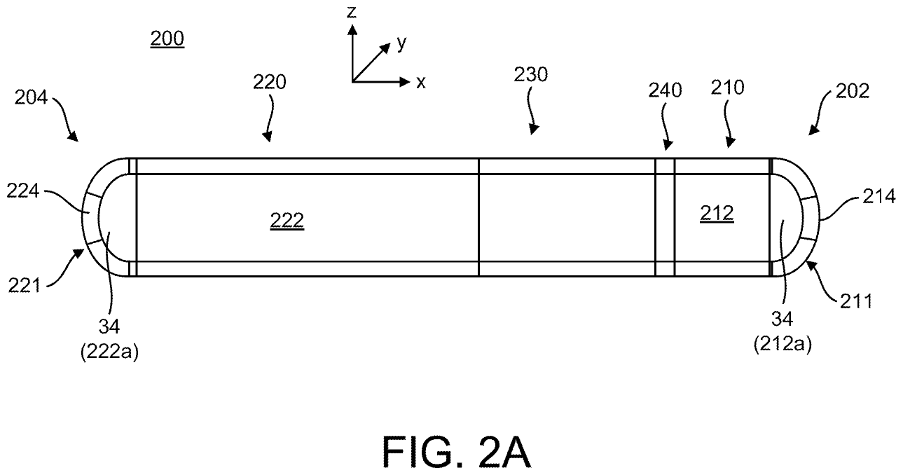

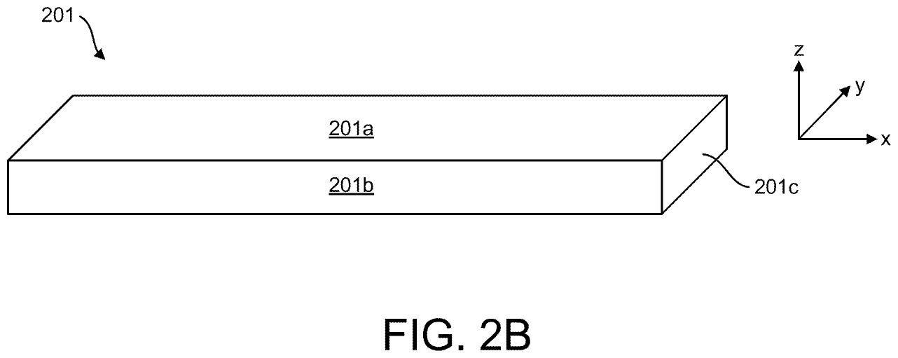

FIG. 2A is a side view of a medical device 200 (hereinafter “IMD 200” for brevity). The IMD 200 may be, or may be similar to, the IMD 102 depicted in FIG. 1 and may be used in the system 100 of FIG. 1.

The IMD 200 includes an external housing that extends between a first end 202 and a second end 204. FIG. 2B is a simplified schematic diagram of an example external housing 201. As shown, the external housing 201 has a general shape of elongate strip with a length, a width, and a thickness along the x, y, and z axes, respectively. The length of the external housing 201 can be generally greater than the width, and greater than the thickness. The external housing 201 includes two opposite major surfaces or faces 201a substantially in the x-y plane, two opposite side surfaces or sides 201b substantially in the x-z plane, and two opposite end surfaces or leading edges 201c substantially in the y-z plane. While a cross-sectional rectangular shape is show in the example schematic diagram of FIG. 2B, it is to be understood that the external housing 201 may have a regular or irregular cross-sectional shape such as, for example, a rectangular shape, an oval shape, a generally round shape, a trapezoid shape, and the like. The adjacent surfaces 201a, 201b, and 201c can be connected by respective a transition portion such as a curved surface, a chamfer, and the like.

In the example of FIG. 2A, the IMD 200 includes a first housing section 210, a second housing section 220, a third housing section 230, and a fourth housing section 240. Although four separate housing sections are shown in FIG. 2A, additional or fewer separate sections can be used to create the IMD 200.

The first housing section 210 is located at or adjacent to the first end 202 and includes a first major surface 212 (e.g., a portion of the major surfaces 201a of FIG. 2B adjacent to the first end 202) and a first end surface or leading edge 214 (e.g., one of the end surfaces 201c of FIG. 2B). In certain instances, the first housing section 210 houses components such as an antenna, recharge coil, portions of a circuit board or flex circuit, and the like. The second housing section 220 is located at or adjacent to the second end 204 and includes a second major surface 222 (e.g., a portion of the major surfaces 201a of FIG. 2B adjacent to the second end 204) and a second end surface or leading edge 224 (another of the end surfaces 201c of FIG. 2B). In certain instances, the second housing section 220 houses a battery (e.g., one or more battery cells such as rechargeable battery cells) and/or other electrical components. Each of the housing sections can be separate components that are assembled together during manufacturing to create the external housing of the IMD 200. When assembled together, the housing sections can create a hermetically sealed enclosure.

As shown, the IMD 200 further includes a first electrode 211 positioned at the first end 202, and a second electrode 221 positioned at the second end 204. The first electrode 211 is wrapped around the first end surface or leading edge 214 at the first end 202 and is referred to as a first end electrode. The second electrode 214 is wrapped around the second end surface or leading edge 214 at the second end 204 and is referred to as a second end electrode. As such, the first electrode 221 and the second electrode 214 are shown as respectively forming the first end 202 and the second end 204 of the IMD 200.

By forming electrodes on opposite end of the IMD 200, the distance between the electrodes is greater than if one of the electrodes was a face electrode (e.g., not positioned at one of the distal ends of the IMD 200). Increasing the distance between the electrodes can improve the quality of the measurements sensed using the electrodes. Further, using two end electrodes increases the overall surface area of the electrodes as compared to if one of the electrodes was a face electrode. Increasing the surface area can further improve the quality of the measurements sensed using the electrodes. As one example, the increased surface area may cause the electrodes to contact different tissue types. Further yet, because ends of the IMD 200 are typically under a more constant pressure load (as compared to an IMD with only one end electrode), the IMD 200 is more likely to be in consistent contact with tissue.

In certain instances, the first electrode 221 and the second electrode 214 comprise one or more conductive materials such as materials comprising titanium, iridium, and the like. In certain instances, the base material (e.g., titanium) has a coating (e.g., to lower impedance).

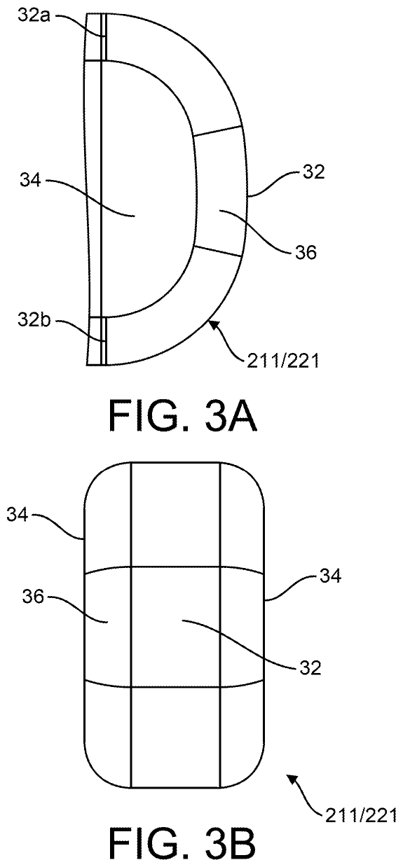

FIGS. 3A and 3B illustrate side views of example first and/or second end electrodes 211 and 221. In this example, each of the first end electrode 211 and the second end electrode 221 includes a curved edge electrode portion 32, a face electrode portion 34, and a transition portion 36 to connect the curved edge electrode portion 32 to the face electrode portion 34. The curved edge electrode portion 32 forms the first or second end surface (e.g., the end surface 214 or 224 of FIG. 2A or the end surface 201c of FIG. 2B) and has the ends 32a and 32b extending onto a side surface (e.g., the side surfaces 201b of FIG. 2B). In the example of FIG. 3A, the curved edge electrode portion 32 forms a semi-oval-shaped profile.

The face electrode portion 34 forms a portion of the first or second major surface 212 or 214 of the first or second housing section 210 or 220 adjacent to the respective ends 202 and 204 (FIG. 2A). For example, as shown in FIG. 2A, the first major surface 212 of the first housing section 210 has an end portion 212a, which forms the face electrode portion 34 of the first end electrode 211. The second major surface 222 of the second housing section 220 has an end portion 222a, which forms the face electrode portion 34 of the second end electrode 221. The transition portion 36 may include a curved surface, a chamfer, and the like to smoothly connect the curved edge electrode portion 32 to the face electrode portion 34 such that the different portions form a mechanically and electrically integral end electrode that is wrapped around the respective end surfaces 214 and 224 (FIG. 2A).

In certain instances, the second end electrode 221 has a larger surface area than the first end electrode 211. Because the first end electrode 211 may be positioned adjacent to components such as an antenna, a recharging coil, etc., there may be less available space for the first end electrode 211 to utilize so that potential interference with such components is limited. As such, the second end electrode 221 may be made larger (e.g., with more surface area) than the first end electrode 211. In certain instances, the second end electrode 221 has a surface area of 70-90 mm² while the first end electrode 211 has a surface area of 60-69.99 mm². In certain instances, the second end electrode 221 has a surface area that is 5-30 mm² greater than the first end electrode 211.

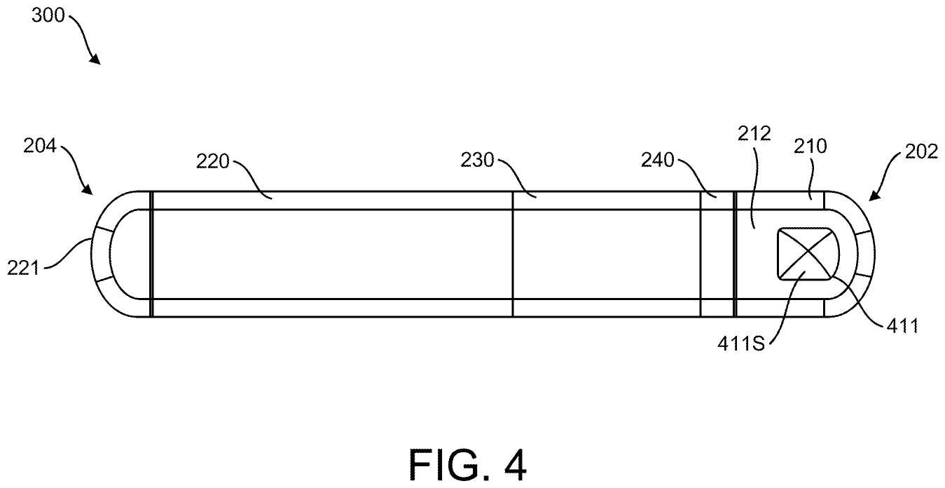

In the example of FIG. 4, the IMD 300 includes one end electrode 221 disposed at the second end 204 and a face electrode 411 coupled to the first major surface 212 of the first housing section 210. The end electrode 221 is wrapped around the second end surface or leading edge at the second end 204 such that the end electrode 221 forms a distal end of the IMD 300.

The face electrode 411 includes an electrode surface 411S extending beyond the first major surface 212 of the first housing section 210. The face electrode 411 can have a total height in a range, for example, from about 2 mm to about 4 mm (e.g., 2-4 mm, 2.5-3.5 mm), as measured from the first major surface 212.

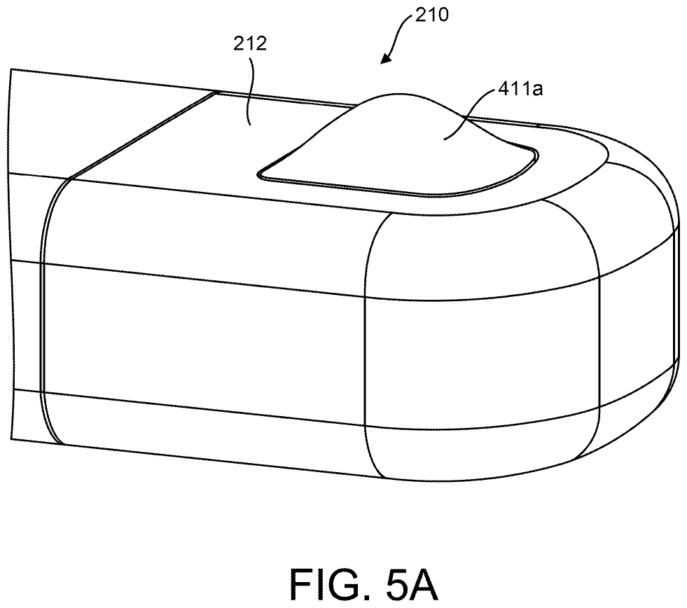

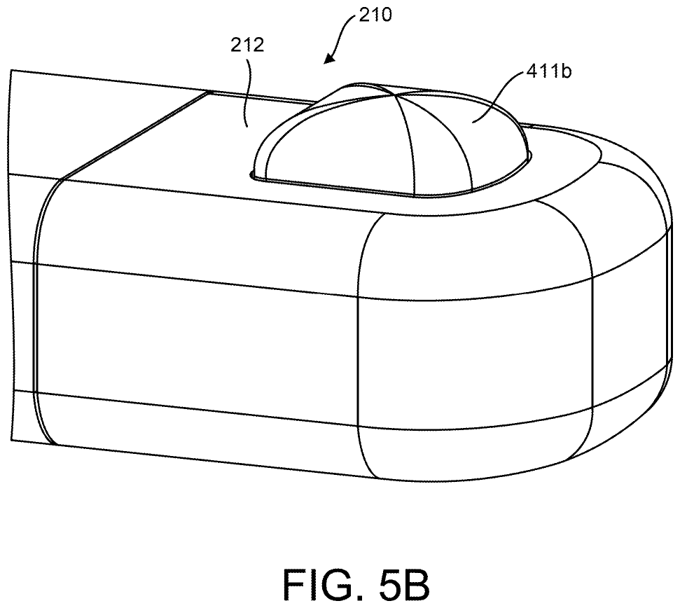

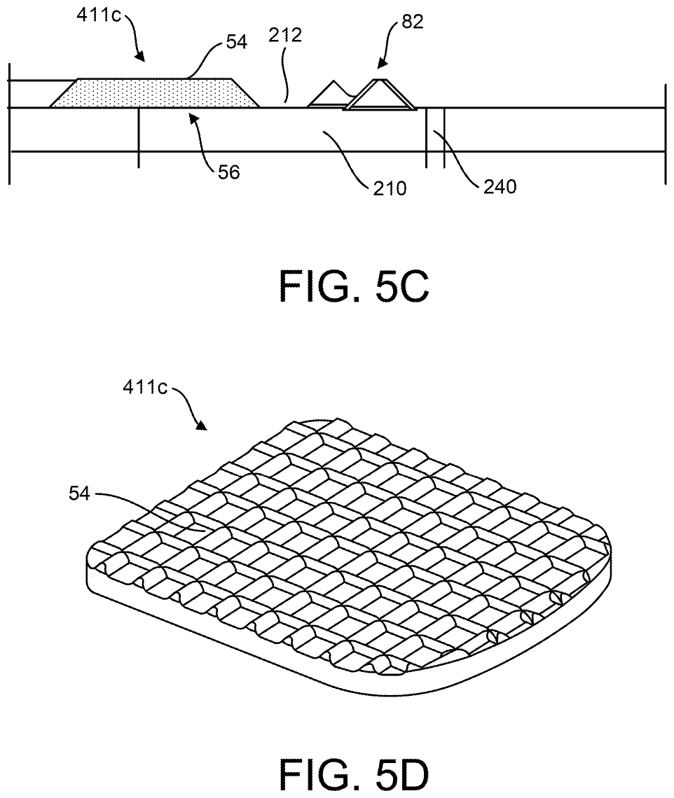

FIGS. 5A-5C show different profiles of the face electrode 411. In the examples of FIGS. 5A and 5B, the face electrode 411a and 411b each have a dome shape. The side profile of the face electrode 411a of FIG. 5A forms a surface with a linear slope (e.g., relative to the first major surface 212) as the face electrode 411a extends from the base (e.g., the first major surface 212) towards a peak (e.g., a central point of the face electrode 411a) and then has a curved transition up to the peak. The side profile of the face electrode 411b of FIG. 5B forms a curved surface that starts with an initial slope (e.g., a steep slope with a 75-90 degree slope) and the slope decreases as the face electrode 411a extends from the base (e.g., the first major surface 212) towards a peak (e.g., a central point of the face electrode 411b).

In the example of FIG. 5C, the face electrode 411c has a truncated cone shape extending between a base level 56 and an outermost surface for direct tissue contact.

FIG. 5D illustrates an electrode surface of the face electrode 411c with a textured surface. A porous surface texture 54 is provided on the face electrode 411c. In the example of FIG. 5D, the porous surface texture 54 includes an array of wells defined by a mesh structure.

Any of the face and end electrodes described herein can include surface texturing (e.g., porous surface texturing). Surface texturing can allow tissue in-growth with the surface area after implantation of the IMD within the patient. A surface texture can be formed on an electrode surface by any suitable methods or processes including, for example, chemical etching, laser texturing, metal additive manufacturing (e.g., 3D metal printing), metal stamping, indenting, extrusion, die forming, forging, rolling, and the like. Examples of a surface texture may include, for example, a protrusion (e.g., a line, a bar, a grid pattern, an island, a granular particle, a woven mesh, and the like), and/or a depression (e.g., an opening, a pore, a hole, a pit, a well, and the like).

The face electrodes described herein may have various shapes such as, for example, a dome shape, a cone shape, a truncated cone shape, a pyramid shape, a truncated pyramid shape, a prism shape, a truncated prism shape, and the like.

In some instances, one or more protrusions can be placed on the outer surface of the IMD to reduce movement of the IMD within an implantation location or pocket in the patient's chest or abdomen. The protrusions can provide a structure that reduces at least one of rotational, translational, and lateral movement of the IMD within a patient's tissue after implantation in the patient. For example, having protrusions arranged at least partially across the width of an IMD may prevent longitudinal movement of the IMD within a patient's tissue, and having protrusions arranged at an angle to the central axis of the IMD may prevent the IMD from flipping or rotating within the patient's tissue. Additionally, having protrusions attached to the outer surface of the IMD may provide a structure that is complementary to the teeth of an insertion or extraction tool, giving a medical provider more control over the IMD while being held. For example, the protrusions may be sized and shaped to provide added frictional engagement with a medical forceps. The protrusions may make it easier for a heath care worker to grab the IMD with an extraction tool and remove the IMD from a patient without the IMD slipping from the extraction tool.

In the example of FIG. 5C, an array of protrusions 82 is provided on the major surface 212 of the first housing section 210, between the face electrode 411c and the fourth housing section 240. The protrusions 82 may have various shapes such as, for example, a truncated triangular prism shape, an elongated truncated pyramid shape, and the like. In some instances, the protrusions 82 may have a height that is comparable to the height of the adjacent face electrode 411c. For example, each of the protrusions 82 and the face electrode 411c can have a height in a range, from 0.5 mm to 5 mm (e.g., 1-4.5 mm, 2-3 mm), as measured from the major surface 212 of the first housing section 210.

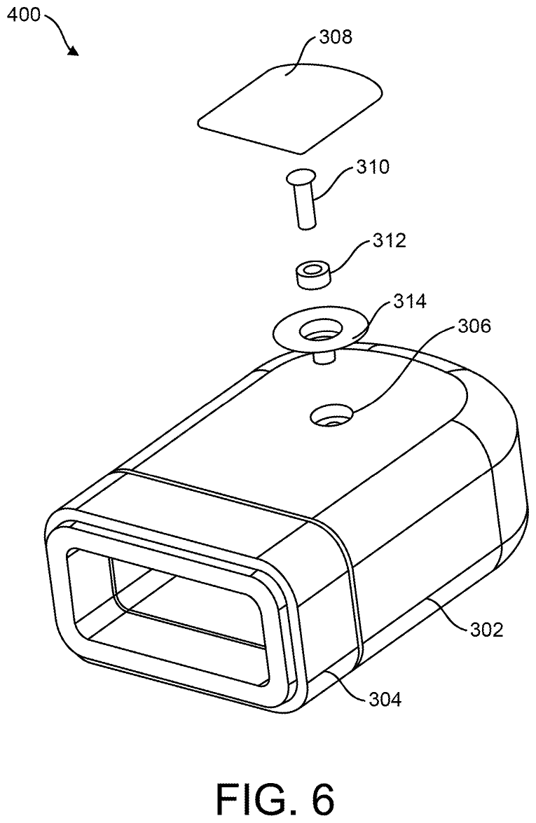

FIG. 6 shows an example of electrical connections between an electrode and a component positioned within the IMD such as a circuit board, flexible circuit, feedthrough, and the like. The approaches shown in FIG. 6 can be used with the IMD 200 or 300. In FIG. 6, a portion of an IMD 400 is shown in an exploded view. The IMD 400 includes a first housing section 302 and a second housing section 304 (e.g., a coupler) connected together and forming an internal cavity or enclosure. In some instances, the first housing section 302 can include a ceramic material. In other instances, the first housing section 302 is an overmolded material such as a polymer.

As shown, an aperture 306 is formed in the first housing section 302. In the illustrated example of FIG. 6, while the aperture 306 is formed in the major surface of the first housing section 302, it is to be understood that the aperture 306 can be formed in an end surface (e.g., 201c in FIG. 2B) or a side surface (e.g., 201b in FIG. 2B) of the first housing section 302. The IMD 300 also includes an electrode assembly that hermetically seals the aperture 306. The electrode assembly includes an electrode 308, a conductor 310 (e.g., conductive pin), and a ring 312. When assembled, the electrode 308 is attached (e.g., adhered, brazed, deposited) to an outer surface.

In some instances, the metallization of the first housing section 302 is represented by component associated with reference number 314. In some instances, a feed-through flange including an aperture can be represented by component associated with reference number 314. The feed-through flange can be received inside first housing section 302 and coupled to a circuit board. The conductor 310 is part of or coupled to the electrode 308 and extends through the aperture 306 in the first housing section 302. The ring 312 represents a brazed joint that helps provide a hermetic seal. The ring 312 can include a material such as gold or palladium, which can be brazed. The conductor 310 can then be coupled to a circuit board (such as those described above) such that the conductor 310 can transmit signals from the electrode 308 and through the first housing section 302 to the circuit board and ultimately to an electrical component such as a microprocessor. In some instances, the conductor 310 includes a pin through the aperture 306 to electrically connect an end electrode or the face electrode to the circuit board section.

### Methods

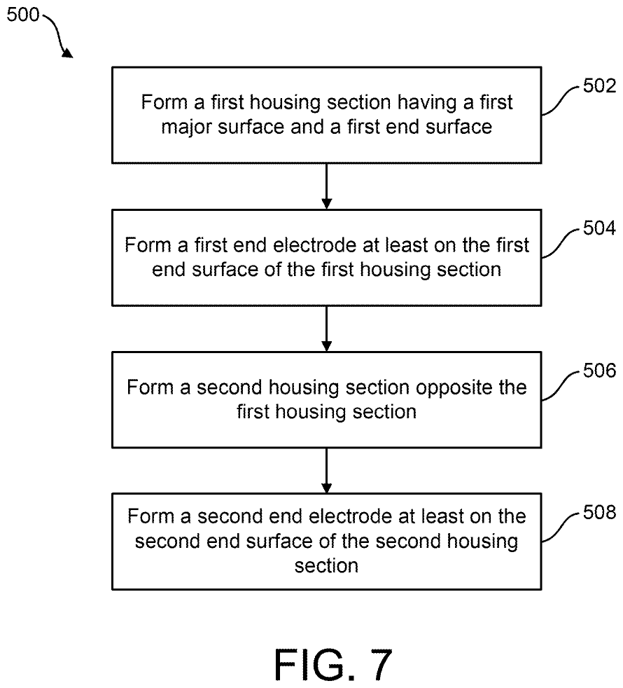

FIG. 7 shows a block diagram of a method 500 of manufacturing the IMDs described herein. The method 500 includes forming a first housing section having a first major surface and a first end surface (block 502). The method 500 further includes forming a first end electrode at least on the first end surface of the first housing section (block 504). The method 500 further includes forming a second housing section opposite the first housing section in a longitudinal direction of the IMD (block 506). The second housing section has a second major surface and a second end surface. The method 500 further includes forming a second end electrode at least on the second end surface of the second housing section (block 508).

In some instances, the IMD extends along the longitudinal direction between a first leading edge and a second leading edge. The first end electrode wraps around the first leading edge. The second end electrode wraps around the second leading edge.

In some instances, the method 500 further includes forming a plurality of porous features on or adjacent to at least one of the first end electrode and the second end electrode.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. An implantable medical device comprising:
a first housing section having a first major surface and a first end surface;
a first end electrode that forms the first end surface;
a second housing section opposite the first housing section in a longitudinal direction of the implantable medical device, the second housing section having a second major surface and a second end surface; and
a second end electrode that forms the second end surface.

2. The implantable medical device of claim 1, further comprising:
a first leading edge; and
a second leading edge,
wherein:
the implantable medical device extends along the longitudinal direction between the first leading edge and the second leading edge,
the first end electrode is wrapped around the first leading edge, and
the second end electrode is wrapped around the second leading edge.

3. The implantable medical device of claim 1, wherein the first end electrode and the second end electrode each comprise:
a curved edge portion;
a face electrode portion to cover at least a portion of the first or second major surface; and
a transition portion to connect the curved edge portion to the face electrode portion.

4. The implantable medical device of claim 3, wherein the transition portion is curved.

5. The implantable medical device of claim 1, wherein the first housing section comprises a ceramic material.

6. The implantable medical device of claim 1, further comprising a plurality of first porous features formed on the first end electrode.

7. The implantable medical device of claim 6, further comprising a plurality of second porous features formed on the second end electrode.

8. The implantable medical device of claim 6, further comprising a plurality of second porous features formed on an area at least partially surrounding at least one of the first end electrode and the second end electrode.

9. The implantable medical device of claim 1, wherein first end electrode and the second electrode comprise titanium.

10. An implantable medical device comprising:
a first housing section having a first major surface and a first end surface;
an end electrode that forms the first end surface;
a second housing section opposite the first housing section in a longitudinal direction of the implantable medical device, the second housing section having a second major surface and a second end surface; and
a face electrode that covers a portion of the second major surface but not the second end surface,
the face electrode forms a shape that protrudes away from the second major surface to increase surface area of the face electrode exposed to tissue.

11. The implantable medical device of claim 10, wherein the dome forms a side profile with a linear slope portion as the face electrode extends from the first major surface towards a peak of the face electrode.

12. The implantable medical device of claim 11, wherein the side profile includes a curved transition between the linear slope portion and the peak.

13. The implantable medical device of claim 10, wherein the dome forms a side profile that continuously curves between the first major surface and a peak of the face electrode.

14. The implantable medical device of claim 13, wherein the side profile has a first slope and a second slope, wherein the second scope decreases as the face electrode extends from the first major surface towards the peak.

15. The implantable medical device of claim 10, wherein the shape comprises a truncated cone shape extending between the first major surface and an outermost surface for direct tissue contact.

16. The implantable medical device of claim 10, further comprising protrusions extending from the second major surface.

17. The implantable medical device of claim 16, wherein the face electrode has a peak at or higher than a distal tip of the protrusions.

18. A method of making an implantable medical device, the method comprising:
forming a first housing section having a first major surface and a first end surface;
forming a first end electrode at least on the first end surface;
forming a second housing section opposite the first housing section in a longitudinal direction of the implantable medical device, the second housing section having a second major surface and a second end surface; and
forming a second end electrode at least on the second end surface.

19. The method of claim 18, wherein:
the implantable medical device extends along the longitudinal direction between a first leading edge and a second leading edge,
the first end electrode is formed to wrap around the first leading edge, and
the second end electrode is formed to wrap around the second leading edge.

20. The method of claim 18, further comprising: forming respective porous surface areas on the first end electrode and the second end electrode.
